(19) United States
(12) Patent Application Publication
(10) Pub. No.: US 2007/0078482 A1
(43) Pub. Date: Apr. 5, 2007

(54) Method and apparatus for pain management

(76) Inventor: Jay Korrol, Royal Palm Beach, FL (US)

Correspondence Address: MCHALE & SLAVIN, P.A., 2855 PGA BLVD, PALM BEACH GARDENS, FL, 33410, US

(21) Appl. No.: 11/243,927

(22) Filed: Oct. 5, 2005

(51) Int. Cl. A61B 17/00

(52) U.S. Cl. 606/204

(57) ABSTRACT

A acupressure band kit for treatment of menstrual cramps includes a flexible strap with a buckle and a free end. A stylus is attached to the buckle for applying pressure to an acupoint on the patient's body when the strap is fastened. Cooperating fasteners are on the free end of the strap and the strap for securing the strap and maintaining pressure on the acupoint. Software is also in the kit which directs the operation of the treatment.

### FIELD OF THE INVENTION

This invention relates to the field of pain management by application of a acupressure device to the body.

### BACKGROUND OF THE INVENTION

Acupuncture has long been an accepted method of treatment in Oriental medicine guided by the principles that the body has meridians in which Qi (energy) flows. Each meridian represents a different organ and each organ represents different aspects and functions. Each meridian has its own set of points and these points also represent different aspects and functions within that meridian's organ. There are certain points in all meridians that have specific functions in common.

By inserting needles at various points in the body along the meridians, the flow of internal energy is redirected bringing relief to the sufferers of various maladies. There are many texts on the subject which teach the location of acupuncture points in the body along with which point is beneficial for treatment of certain symptoms. By inserting the needles at specific points, a particular malady or its symptoms can be successfully treated. There has been a lack of general acceptance of acupuncture resulting from skepticism regarding the theory and reluctance to undergo the needle punctures with the attendant possibility of localized pain and infection.

It has now been determined that acupressure which teaches merely applying external pressure to the acupuncture points on the body can have the same or similar results without the need of needle punctures. Such treatment clearly overcomes a patient's fear of needle sticks and infection.

The skilled practitioner of acupuncture/acupressure is trained in the diagnosis required by this 2000 year old art and the selection of the treatment site on the patient's body that will produce beneficial effects for the diagnosed malady. There are a great many acupoints on the body, the location of each usually described in anatomical terms. The art in the practice is the selection of the proper acupoint or acupoints to use when a malady involves more than one organ and meridian. The skilled artisan can precisely locate these points to bring the full effect of the treatment to bear accurately and squarely on the selected point. The patient's relief may be immediate. In syndromes with recurring cycles, such as problems associated with menstruation, repeated acupressure treatment may be required at the onset of each episode of symptoms.

One of the sets of points in all meridians is known as, “Accumulation Points.” Each meridian has one. The main function of these points is for, “acute,” (sudden onset) patterns accompanied by pain. The particular point of interest here is the accumulation point along the Spleen meridian, specifically the 8th (Sp 8) of the 21 points that belong to the Spleen meridian.

There have been attempts to bring the benefits of acupressure to the public through self-help publications and apparatus to be applied to various parts of the body.

### DESCRIPTION OF THE PRIOR ART

U.S. Pat. No. 6,282,443 issued Aug. 28, 2001 to Mann et al teaches a wrist band which applies an electric current to the body to treat menstrual cramps.

U.S. Pat. No. 5,701,608 issued Dec. 30, 1997 to Kohn teaches an undergarment brief with a pressure insert mounted on an elastic panel for applying pressure to the abdominal area to relieve menstrual cramps.

U.S. Pat. No. 4,479,495 issued Oct. 30, 1984 to Isaacson teaches an elastic band with Velcro fasteners carrying a flexible protuberance to be applied about different parts of the body, as the consumer sees fit, so that the protuberance contacts an acupoint. To use the device, the consumer would have to be skilled in the Chinese diagnoses of a malady, make the correct diagnoses, and correctly select one acupoint to treat the diagnosed malady.

U.S. Pat. No. 6,711,750 issued Mar. 30, 2004 to Yoo teaches the use of an elastic belt with adjustable acupressure pads, in addition to heat pads, for placement about the waist of a user. The belt may have Velcro fasteners for connecting the ends together.

U.S. Pat. No. 5,792,176 issued Aug. 11, 1998 to Chang teaches the use of an adhesive pad supporting a rigid nub for applying pressure to a selected point on the body.

What the prior art lacks is a teaching of a specific apparatus to be applied to a specific location to treat a specific malady accompanied with the clear instructions of how and where the consumer is to apply the apparatus.

### SUMMARY OF THE INVENTION

Accordingly, it is a primary objective of the instant invention to teach the construction of an acupressure band carrying a stylus for applying pressure to an acupoint on the body of a patient.

It is a further objective of the instant invention to teach the use of a kit containing an acupressure band with detailed instructions informing the consumer, unskilled in the acupressure art, to correctly place and apply the band.

It is yet another objective of the instant invention to teach the construction of an acupressure band wherein the stylus is located for maximum sustained pressure.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective of the acupressure band of this invention;

FIG. 2 is a cross section of the acupressure band and buckle of this invention;

FIG. 3 is a cross section of the stylus of this invention;

FIG. 4 is a top plan view of the stylus and buckle of this invention; and

FIG. 5 is a representation of acupoint Sp 8 of this invention.

### DETAILED DESCRIPTION OF THE INVENTION

The acupressure band 10, shown in FIGS. 1-4, has a flexible strap portion 11, a buckle 12 and a stylus 13. The buckle 12 has an aperture 14 through which the free end 15 of the band passes to engage the Velcro fastener 16 on the outside surface 17 of the strap portion 11. The inside surface 18 of the free end 15 has a cooperating patch of Velcro fastener 19 attached thereto.

Velcro is a trademark for a fastening material consisting of two cooperating swatches of material. One swatch has a series of small loops formed on one surface. The other swatch has a series of upstanding hooks on one surface. When the two surfaces are pressed together, the hooks and loops engage to form a releasable connection.

The flexible band portion 11 may be formed of a solid plastic ribbon which is flexible enough to be coiled on itself but not necessarily resilient to prevent stretching when the band is placed on the patient's body under tension. Other materials may be used, such as textiles or metal or plastic meshes, to form a band with the requisite properties.

The buckle 12 may be formed as a unitary extension of the strap 11 or as a separate component permanently affixed to the band. The stylus 13 is molded or otherwise formed on the inside surface of the buckle. Alternatively, the base 20 of the stylus may be adhered to the surface of the buckle by autologous bonding using heat and pressure or the use of compatible adhesives.

The free end of the buckle is formed as a tab 24 to facilitate the user grasping the buckle in one hand. The free end 15 of the band is inserted through the slot 14 in the buckle and the user grasps the end of the band with the other hand. By moving the hands apart, the circumference of the band is reduced applying pressure to the encircled body part of the patient and applying tension to the band. By locating the stylus immediately adjacent the overlap of the ends of the belt, the amount of compression is greatest and can be accurately controlled by adjusting the band length. When the desired amount of pressure is generated by the band, the Velcro swatch on the free end of the band is brought into contact with the Velcro swatch on the outside surface of the band to fix the band in place, as shown in FIG. 2. Of course, other releasable fasteners may be mounted on the band.

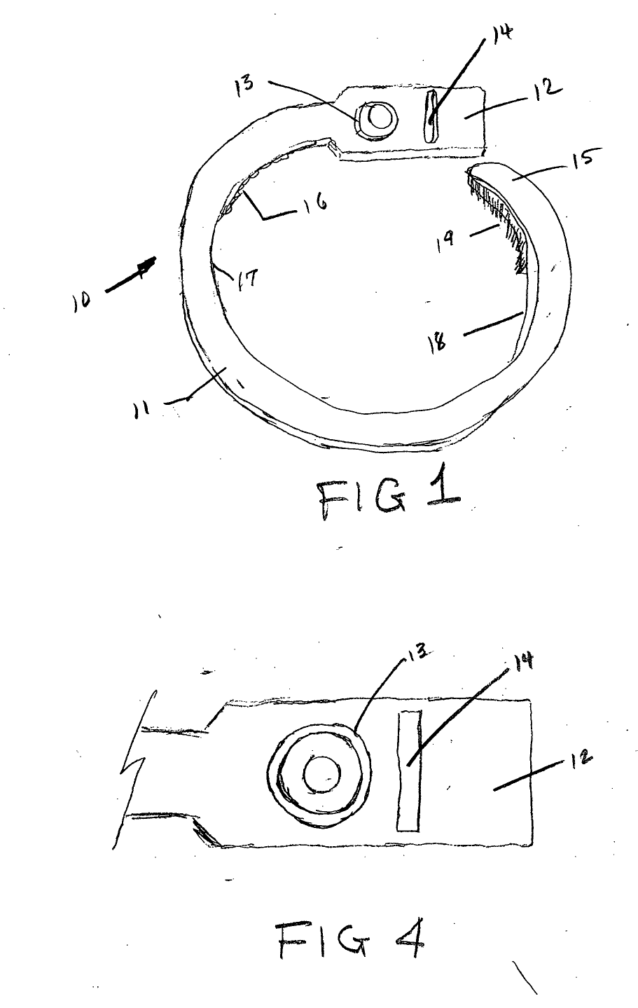

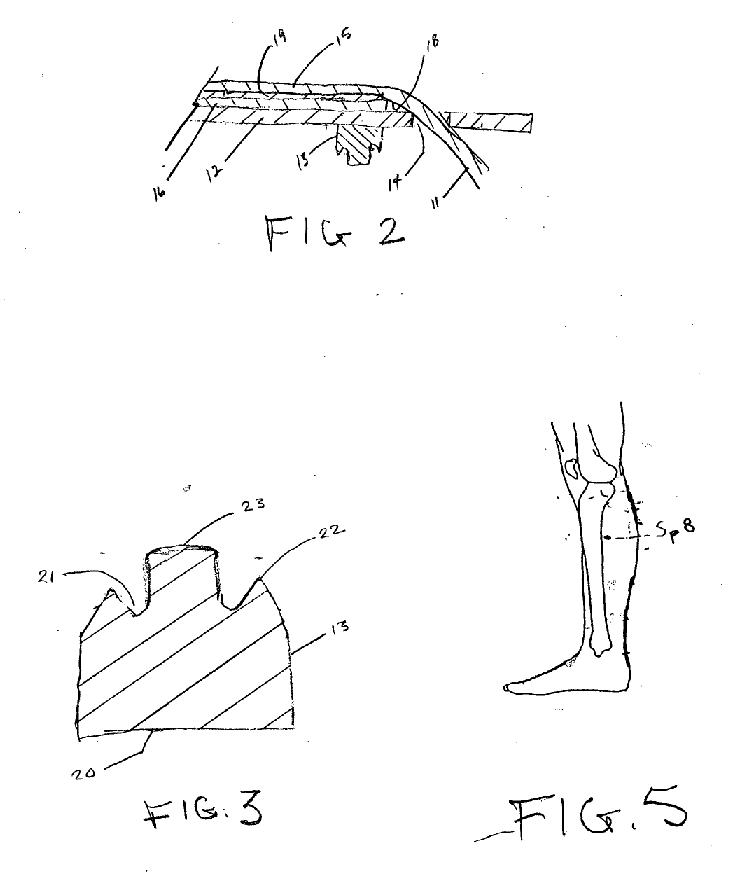

The stylus 13, shown in FIGS. 3 and 4, may be formed of plastic, carbon fibers, metal, ceramic, wood, bone or other suitable material compatible with skin contact. The stylus is designed to apply pressure to a point on the patient's body that produces the equivalent result of acupuncture. The base 20 is permanently connected to the buckle 12 and is of sufficient diameter to stabilize the stylus perpendicular to the buckle. The stylus tapers from said base toward a smaller distal end. The stylus has a peripheral relieved ring 21 with a surrounding ridge 22 near the distal end. The distal end of the stylus extends from the inner surface of the relieved ring beyond the ridge 22 and terminates in a small, smooth end 23. The end should be of such a size that it will not penetrate the skin when the band is tightened to the optimum pressure but will transmit a concentrated force on the acupressure point. The relieved ring 21 and ridge 22 serve to tighten the patient's skin around the stylus and flatten the underlying tissue to concentrate the pressure on the acupressure point. The skin and underlying tissue partially fills the relieved ring to provide resistence to movement of the stylus either by muscular movement of the patient or external forces.

The acupressure band is supplied with software necessary to the proper use of the device. The software may be in the form of a printed brochure, an audio tape, a floppy disk, or video disk (DVD) accompanying the band and forming a kit. The patient is instructed in the theory of acupressure, the anatomical location of the particular acupoint that relates to the malady to be treated is shown in illustrations or video, as well as, the correct amount of force to be used and the time frame of treatment.

### EXAMPLE

Menstrual cramps are a recurring painful malady associated with the menstrual cycle. The acupressure band of this invention can provide relief from this malady. The acupressure point for this treatment is Sp 8. As shown in the software, the location of Sp 8 is approximately 3 cun (inches) below the lower border of the medial condyle of the tibia and approximately 1 inch from the edge of the tibia, as shown in FIG. 5. The measurements are approximate because the locations will be different for different sized women.

As shown by the software, the proper method of determining the location of Sp 8, the acupoint, is by the patient's own proportions. 3 cun is the distance between the index finger and the little finger on each hand. To identify Sp 8, the patient uses their own hand. Place the right hand on the inside of the left calf with the right index finger at the lower border of the medial condyle of the tibia. The little finger rests on the approximate position of Sp 8, as shown in FIG. 5. To authenticate the Sp 8, fine tune the location of Sp 8, to locate the center of the acupoint, by pressing on different spots in this area to find the spot that is most sensitive to the pressure. There may be more than one such spot but select the most sensitive. Apply the stylus to this spot and fix the band around the leg. Tighten the band to a level that is tolerable, without causing pain, and secure the fasteners. The cramps should begin to diminish and disappear.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

I claim:

1. An acupressure band for applying pressure to an acupressure point to treat menstrual cramping comprising an elongated flexible strap for encircling a patient's limb under tension, said strap having a buckle at one end and a free end adapted to pass through said buckle forming a loop in said strap for encircling a limb, said loop having an outside surface and an inside surface, cooperating fasteners on said outside surface and on said inside surface adapted to adjustably and releasably secure said loop about the limb, a stylus attached to said buckle and projecting therefrom toward said inside surface of said strap, said stylus having a distal end adapted to contact the acupoint whereby said loop may be reduced to apply pressure through said stylus to the acupoint.

2. An acupressure band of claim 1 comprising a base on said stylus opposite said distal end, said based connected to said buckle, a relieved ring about the circumference of said stylus between said base and said distal end, said relieved ring defined by a ridge, said relieved ring adapted to engage the limb and stabilize said stylus in position.

3. An acupressure band of claim 2 comprising said stylus tapered from said base toward a smaller distal end, said relieved ring of larger diameter than said distal end.

4. An acupressure band of claim 1 comprising said buckle having a slot for said free end of said strap, said slot adjacent to said stylus, a tab extending from said slot opposite said stylus and adapted to be grasped by the patient.

5. An acupressure band of claim 1 comprising a swatch of Velcro fastener on said inside surface of said free end, a swatch of cooperating Velcro fastener on said outside surface of said strap whereby said strap is releasably secured when said Velcro fastener is connected.

6. A acupressure kit for applying pressure to an acupressure point to treat menstrual cramping comprising an acupressure band and software for correctly applying said band, said band including an elongated strap with a first free end and a second end having a buckle, a stylus mounted on said buckle, said stylus having a distal end adapted to contact the Sp 8 acupressure point on a patient's body, a slot in said buckle adjacent said stylus adapted to pass said free end of said strap, Velcro fastener on said free end and said strap for securing said band in a loop about a patient's limb, said software demonstrating the location of the Sp 8 acupressure point, identifying and authenticating the correct location and instructing in the use of the band to deliver proper application of pressure.

7. A acupressure kit for applying pressure to an acupressure point of claim 6 comprising said stylus being tapered from said buckle to a smaller distal end, a relieved ring about said stylus defined by an upstanding ridge between said buckle and said distal end, said relieved ring adapted to create a flattened area about said stylus.

8. A method of use of a acupressure band in the treatment of menstrual cramps comprising the steps of
(a) providing an acupressure band comprising an elongated strap having a buckle at one end and a free end, a stylus mounted on said buckle with a distal end for contact with an acupoint on the patient's body, a slot in said buckle adjacent said stylus for passing said free end, cooperating fasteners on said free end and said strap for securing said band in a loop;
(b) providing software for operation of said band, said software including instructions indicating the location of use on the patient's body, identifying the proper spot, authenticating the proper spot and determining the correct amount of pressure to apply;
(c) using the hand to identify the location of the Sp 8 acupoint as directed by said software;
(d) authenticating the center of the acupoint as directed by said software;
(e) placing said band about the leg of the patient encircling the leg;
(f) contacting Sp 8 with said stylus and holding stylus in position;
(g) passing said free end of said strap through said slot;
(h) tightening said strap to apply pressure to Sp 8 by said stylus to the level as directed by said software; and
(i) securing said strap by said cooperating fasteners.
